(19) United States
(12) Patent Application Publication
(10) Pub. No.: US 2013/0324513 A1
(43) Pub. Date: Dec. 5, 2013

(54) METHOD FOR TREATING ESOPHAGEAL CANCER

(71) Applicant: Niiki Pharma Acquisition Corp. 2, Tampa, FL (US)

(72) Inventor: Hooshmand Sheshbaradaran, Hoboken, NJ (US)

(73) Assignee: Niiki Pharma Acquisition Corp. 2, Tampa, FL (US)

(21) Appl. No.: 13/958,833

(22) Filed: Aug. 5, 2013

Related U.S. Application Data
(63) Continuation of application No. 13/663,432, filed on Oct. 29, 2012.
Application No. 13/663,432 is a continuation of application No. PCT/US2011/034408, filed on Apr. 29, 2011.
(60) Provisional application No. 61/329,369, filed on Apr. 29, 2010.

(51) Int. Cl. A61K 31/555

(52) U.S. Cl. CPC A61K 31/555
USPC 514/187

(57) ABSTRACT

Methods and compositions for treating gastric and esophageal cancers are disclosed.

### CROSS-REFERENCE TO RELATED U.S. APPLICATIONS

This application is a continuation of U.S. patent application Ser. No. 13/663,432, filed on Oct. 29, 2012; which is a continuation of PCT/US11/34408 filed Apr. 29, 2011, which claims the benefit of U.S. Provisional Application No. 61/329,369 filed on Apr. 29, 2010, the entirety of each is hereby incorporated by reference.

### FIELD OF THE INVENTION

The present invention generally relates to pharmaceutical compositions and methods for treating cancer, and particularly to a pharmaceutical composition and method for treating gastric and esophageal cancers.

### BACKGROUND OF THE INVENTION

Gastric cancer is one of the most deadly forms of cancer. Treatment option for gastric cancer has been limited. Surgery and radiation therapy can be used for early-stage gastric cancer, but not very effective for advanced or recurrent gastric cancer. Traditional chemotherapeutic agents such as 5-fluorouracil and cisplatin have shown very limited effect often causing serious side effects. Thus, there is a significant unmet need for new agents and methods for treating gastric cancer.

Tris(8-quinolinolato)gallium(III) is an organic gallium complex that has been suggested to be useful in certain types of cancer. For example, U.S. Pat. No. 7,919,486 discloses and claims the use of tris(8-quinolinolato)gallium(III) and related compounds for the treatment of melanoma.

### SUMMARY OF THE INVENTION

The present invention provides methods of treating gastric cancer and esophageal cancer. In one aspect, the present invention provides a method of treating, preventing or delaying the onset of, gastric cancer and esophageal cancer comprising administering to a patient having gastric cancer or esophageal cancer a therapeutically or prophylatically effective amount of a compound according to Formula (I) below or a pharmaceutically acceptable salt thereof (e.g., tris(8-quinolinolato)gallium(III)).

Use of the compound according to Formula (I) below or a pharmaceutically acceptable salt thereof (e.g., tris(8-quinolinolato)gallium(III)) for the manufacture of a medicament for use in the methods of the present invention is also provided.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a graph showing the dose-dependent growth inhibition by tris(8-quinolinolato)gallium(III) in an MTT assay in human gastric cancer cell line NCI-N87. X axis is drug concentration in nM and Y axis is percentage of control in absorbance;

FIG. 2 is a graph showing the dose-dependent growth inhibition by tris(8-quinolinolato)gallium(III) in an MTT assay in human esophageal carcinoma cell line OE33. X axis is drug concentration in nM and Y axis is percentage of control in absorbance.

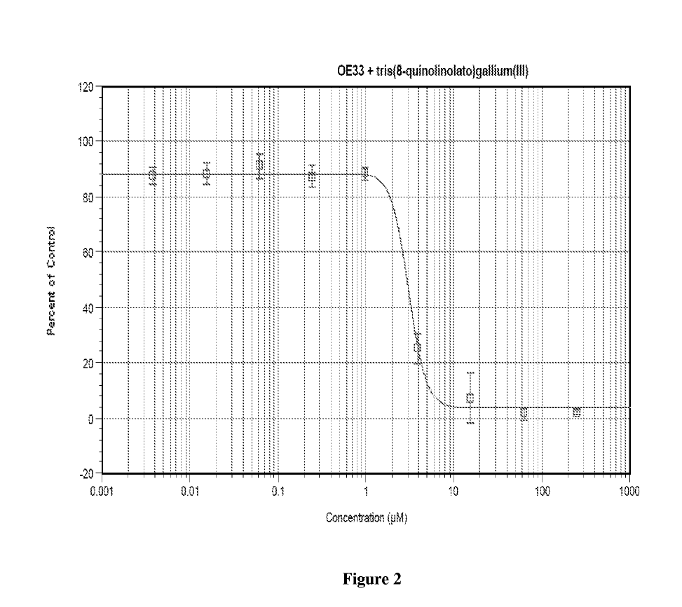

### DETAILED DESCRIPTION OF THE INVENTION

The present invention is at least in part based on the discovery that the compound tris(8-quinolinolato)gallium(III) is effective in treating gastric cancer and esophageal cancer. Accordingly, in accordance with a first aspect of the present invention, a method is provided for treating gastric cancer and esophageal cancer. The method comprises treating a gastric or esophageal cancer patient in need of treatment with a therapeutically effective amount of a gallium complex of Formula (I)

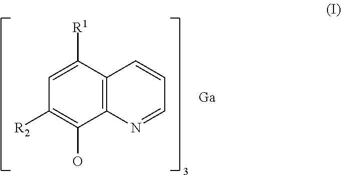

wherein R¹ represents hydrogen, a halogen or a sulfono group SO₃M, in which M is a metal ion, and R² represents hydrogen, or R¹ is Cl and R² is I, or a pharmaceutically acceptable salt thereof. In one embodiment, the method for treating gastric or esophageal cancer comprises treating a gastric or esophageal cancer patient in need of treatment with a therapeutically effective amount of compound of Formula (I) or a pharmaceutically acceptable salt thereof. That is, the present invention is directed to the use of an effective amount of a compound according to Formula (I) or a pharmaceutically acceptable salt thereof for the manufacture of medicaments for treating a gastric or esophageal cancer in patients identified or diagnosed as having a gastric or esophageal cancer.

In preferred embodiments, the compound according to Formula (I) is tris(8-quinolinolato)gallium(III)

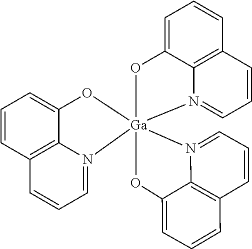

or a pharmaceutically acceptable salt thereof.

The method of the present invention can be useful in various gastric and esophageal malignancies including, but not limited to, gastric adenocarcinoma (intestinal type or diffuse type), MALT lymphoma (MALToma), stromal tumors, gastrointestinal stromal tumor (GIST), esophageal squamous cell carcinomas, esophageal adenocarcinomas, leiomyoma, and small-cell carcinomas.

In the various embodiments of this aspect of the present invention, the treatment method optionally also comprises a step of diagnosing or identifying a patient as having gastric or esophageal tumor. The identified patient is then treated with or administered with a therapeutically effective amount of a compound of the present invention, e.g., tris(8-quinolinolato)gallium(III). Various gastric or esophageal cancers can be diagnosed in any conventional diagnostic methods known in the art including CT scan, endoscopy, barium roentgenogram, biopsy, etc.

In accordance with yet another aspect of the present invention, a method is provided for preventing or delaying the onset of gastric and esophageal cancer, or preventing or delaying the recurrence of gastric and esophageal cancer, which comprises treating a patient in need of the prevention or delay with a prophylatically effective amount of a compound of Formula (I) or a pharmaceutically acceptable salt thereof (e.g., tris(8-quinolinolato)gallium(III)).

For purposes of preventing or delaying the recurrence of gastric or esophageal cancer, gastric or esophageal cancer patients who have been treated and are in remission or in a stable or progression free state may be treated with a prophylatically effective amount of a compound of Formula (I) or a pharmaceutically acceptable salt thereof (e.g., tris(8-quinolinolato)gallium(III)) to effectively prevent or delay the recurrence or relapse of gastric or esophageal cancer.

As used herein, the phrase “treating . . . with . . . ” or a paraphrase thereof means administering a compound to the patient or causing the formation of a compound inside the body of the patient.

In accordance with the method of the present invention, gastric or esophageal cancer can be treated with a therapeutically effective amount of a compound of Formula (I) or a pharmaceutically acceptable salt thereof (e.g., tris(8-quinolinolato)gallium(III)) alone as a single agent, or alternatively in combination with one or more other anti-cancer agents.

The pharmaceutical compounds of Formula (I) can be administered through intravenous injection or oral administration or any other suitable means at an amount of from 0.1 mg to 1000 mg per kg of body weight of the patient based on total body weight. The active ingredients may be administered at predetermined intervals of time, e.g., three times a day. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In accordance with the present invention, it is provided a use of a compound having a compound of Formula (I) or a pharmaceutically acceptable salt thereof (e.g., tris(8-quinolinolato)gallium(III)) for the manufacture of a medicament useful for treating gastric and esophageal cancers. The medicament can be, e.g., in an oral or injectable form, e.g., suitable for intravenous, intradermal, or intramuscular administration. Injectable forms are generally known in the art, e.g., in buffered solution or suspension.

In accordance with another aspect of the present invention, a pharmaceutical kit is provided comprising in a container a unit dosage form of a compound of Formula (I) or a pharmaceutically acceptable salt thereof (e.g., tris(8-quinolinolato)gallium(III)), and optionally instructions for using the kit in the methods in accordance with the present invention, e.g., treating, preventing or delaying the onset of gastric or esophageal cancer, or preventing or delaying the recurrence of gastric or esophageal cancer. As will be apparent to a skilled artisan, the amount of a therapeutic compound in the unit dosage form is determined by the dosage to be used on a patient in the methods of the present invention. In the kit, a compound having a compound of Formula (I) or a pharmaceutically acceptable salt thereof (e.g., tris(8-quinolinolato)gallium(III)) can be in a tablet form in an amount of, e.g., 1 mg.

### EXAMPLE 1

To test the activities of tris(8-quinolinolato)gallium(III), ATCC's MTT Cell Proliferation Assay® was performed using human gastric cancer cell line NCI-N87 (differentiated carcinoma). Stock cultures were allowed to grow to 70-80% confluence for this study. The anti-proliferative activity of tris(8-quinolinolato)gallium(III), against the indicated cell line was evaluated in vitro using the the ATCC's MTT Cell Proliferation Assay (Catalog No. 30-1010K). NCI-N87 was grown using RPMI1640 (Cell Gro 10-040-CV), with 1% of 1M HEPES, 1% sodium pyruvate, 1% of 45% glucose solution, 10% of heat-inactivated FBS and 1% of pen/strep/glutamine. NCI-N87 cell plates were seeded with 20E+03 cells/well and treated with tris(8-quinolinolato)gallium(III) at 1,000 μM, or a series of 4×dilutions thereof (250 μM, 62.5 μM, etc.). 100 μl of medium was removed from each well at 72 hours post-treatment and 10 μl MTT reagent was added to each well. The plates were incubated plate at 37° C. for 4 hours and then 100 μl of detergent was added. The plates were left overnight at room temperature in the dark and was read on a plate reader using SoftMax® Pro (version 5.2, Molecular Devices).

The absorbance data was analyzed as follows: Absorbance values were converted to Percent of Control and plotted against test agent concentrations for IC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration versus Percent of Control were analyzed using the 4-parameter equation to obtain IC₅₀ values and other parameters that describe the sigmoidal dose response curve.

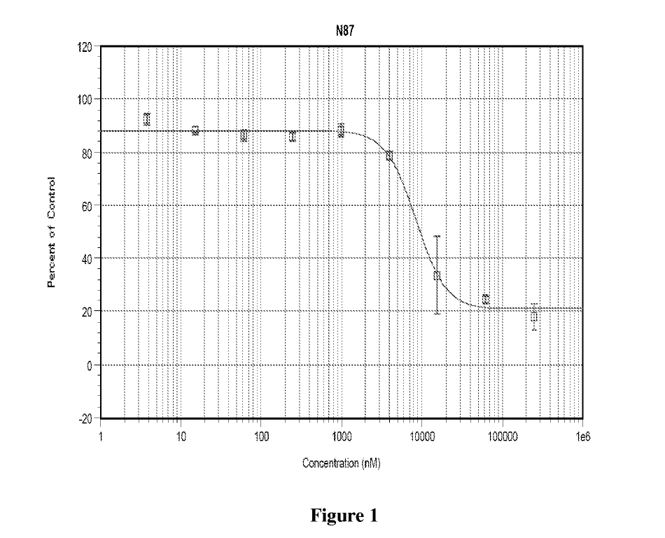

The IC₅₀ value for the test agents was estimated by curve-fitting the data using the following four parameter-logistic equation:

$$Y = \frac{Top - Bottom}{1 + {( \frac{X}{{IC}_{50}} )}^{n}} + Bottom$$

wherein “Top” is the maximal % of control absorbance (100%) (Value “A” in FIG. 1, 88.3), “Bottom” is the minimal % of control absorbance at the highest agent concentration (down to zero) (Value “D” in FIG. 1, 20.8), Y is the Percent of Control absorbance, X is the test agent Concentration, IC₅₀ is the concentration of agent that inhibits cell growth by 50% compared to the control cells (Value “C” in FIG. 1, 8.42e+03), n is the slope of the curve (Value “B” in FIG. 1, 2.31). The IC₅₀ of tris(8-quinolinolato)gallium(III) in NCI-N87 cell line was 8.42 μM.

### EXAMPLE 2

To test the activity of tris(8-quinolinolato)gallium(III), ATCC's MTT Cell Proliferation Assay® was performed using human esophageal carcinoma cell line OE33. Stock cultures were allowed to grow to 70-80% confluence for this study. The anti-proliferative activity of tris(8-quinolinolato)gallium(III) against the indicated cell line was evaluated in vitro using the ATCC's MTT Cell Proliferation Assay (Catalog No. 30-1010K). OE33 plates were seeded with 1,200 cells/well, and the cells were grown in RPMI1640 medium containing 1% (1M HEPES), 1% sodium pyruvate, 10% FBS and 1% penicillin/strep/glutamine. Cultures were maintained in a 37° C. humidified 5% CO₂/95% air atmosphere. The cells were treated with tris(8-quinolinolato)gallium(III) at 1,000 μM, or a series of 4×dilutions thereof (250 μM, 62.5 μM, etc.). 100 μl of medium was removed from each well at 72 hours post-treatment and 10 μl MTT reagent was added to each well. The plates were incubated at 37° C. for 4 hours and then 100 μl of detergent was added. The plates were left overnight at room temperature in the dark and was read on a plate reader using SoftMax® Pro (version 5.2, Molecular Devices).

The absorbance data was analyzed as follows: Absorbance values were converted to Percent of Control and plotted against test agent concentrations for IC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration versus Percent of Control were analyzed using the 4-parameter equation to obtain IC₅₀ values and other parameters that describe the sigmoidal dose response curve.

The IC₅₀ value for the test agent was estimated by curve-fitting the data using the following four parameter-logistic equation:

$$Y = \frac{Top - Bottom}{1 + {( \frac{X}{{IC}_{50}} )}^{n}} + Bottom$$

wherein “Top” is the maximal % of control absorbance (100%), “Bottom” is the minimal % of control absorbance at the highest agent concentration (down to zero), Y is the Percent of Control absorbance, X is the test agent Concentration, IC₅₀ is the concentration of agent that inhibits cell growth by 50% compared to the control cells, n is the slope of the curve. The IC₅₀ of tris(8-quinolinolato)gallium(III) in the OE33 cell line was 3.06 μM.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. A method for treating gastric or esophageal cancer, comprising administering to a patient in need of treatment, a compound of Formula (I) 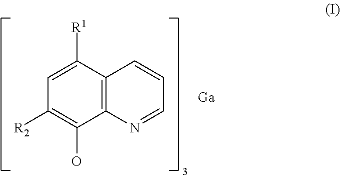
wherein R¹ represents hydrogen, a halogen or a sulfono group SO₃M, in which M is a metal ion, and R² represents hydrogen, or R¹ is Cl and R² is I, or a pharmaceutically acceptable salt thereof.

2. The method of claim 1, said compound is tris(8-quinolinolato)gallium(III).

3. The method of claim 1, wherein the patient is diagnosed of gastric cancer.

4. The method of claim 1, wherein the patient is diagnosed of esophageal cancer.
